(19) United States
(12) Patent Application Publication
(10) Pub. No.: US 2009/0042158 A1
(43) Pub. Date: Feb. 12, 2009

(54) Method for regenerating bone in the maxillary sinus

(76) Inventor: Gregory Gene Steiner, Kapolei, HI (US)

Correspondence Address: GREGORY GENE STEINER, 590 FARRINGTON HWY. #524, SUITE 132, KAPOLEI, HI, 96707, US

(21) Appl. No.: 11/891,579

(22) Filed: Aug. 9, 2007

(51) Int. Cl. A61C 3/00; A61B 17/58

(52) U.S. Cl. 433/7; 606/87

(57) ABSTRACT

The present invention relates to a method of treating bone loss in the posterior maxilla. Adequate bone is required in the posterior maxilla to facilitate tooth replacement with dental implants. The Lateral wall of the maxilla is exposed and an osteotomy is performed to expose the sinus membrane. Pneumatic pressure is utilized to detach the membrane from the lateral wall of the sinus. Once detached fluid bone graft material is injected between the bone of the maxilla and the sinus membrane thereby lifting the membrane with hydraulic pressure. The combination of pneumatic pressure to detach the membrane and hydraulic pressure to create a void for grafting material presents numerous benefits to the patient and surgeon.

### FIELD OF THE INVENTION

Bone graft surgery is a common procedure in both medicine and dentistry. Bone grafts are used to regenerate bone lost due to trauma, disuse or disease. The unfortunate removal of a tooth precipitates an initial loss of alveolar bone due to exposure of alveolar bone to the oral environment and disuse atrophy. One of the greatest threats to the patient is osteomyelitis. Consequentially the body has developed a potent response to exposure of bone in order to protect the patient from infection. This response results in resorption or rejection of damaged bone. After the initial healing of exposed bone the alveolar ridge gradually resorbs. In addition as we age our maxillary sinuses increase in size. The combination of resorption of the alveolar ridge and pneumatization of the maxillary sinuses results in inadequate bone for dental implants. The bone lost in the posterior maxilla due to resorption of the alveolar ridge and pneumatization of the sinus requires bone regeneration prior to the replacement of teeth with dental implants.

### BACKGROUND OF THE INVENTION

Over the years various methods and materials have been devised to rebuild the bone in the maxillary posterior alveolar ridges in order to provide enough bone for dental implants. The most common method for regenerating bone in the posterior maxilla is to perform a sinus lift. However, traditionally these methods are invasive resulting is significant post operative morbidity. The common method is to cut a window into the lateral wall of the sinus without damaging the sinus membrane and pack in granular bone graft material. This required a large osteotomy of the lateral wall of the maxillary sinus in order to visualize the sinus membrane and pack in hard bone graft material that fills the bottom of the sinus. This method results in significant post operative morbidity. A method designed to limit the post operative morbidity of the sinus lift procedure is to perform an osteotomy in the crest of the alveolar bone where the implants are to be placed and fracturing in the bottom of the floor of the sinus. After the floor of the sinus bone was fractured up hard bone graft material is packed into the osteotomy and into the floor of the sinus. However, due to the inability to see the sinus membrane during the fracturing and packing procedure this method commonly resulted in damaging the delicate sinus membrane resulting in the inability to successfully complete the sinus augmentation. A third method described by Chen used the same crestal osteotomy approach but rather than fracturing up the floor of the sinus the osteotomy is continued until the membrane is exposed. Then the membrane is separated from the floor of the sinus by water under hydraulic pressure. After water was forced into the sinus under hydraulic pressure the traditional lateral osteotomy window is cut into the lateral wall of the sinus and hard bone graft material is packed into the sinus. This method suffers from the same difficulty as the sinus membrane is exposed by way of the crestal implant osteotomy which cannot be visualized and commonly results in damage to the membrane. In addition this method requires an additional osteotomy in the lateral wall of the sinus to pack in hard bone graft material. The method described by Chen (http://www.osseonews.com/hydraulic-sinus-condensing-technique) uses hydraulic pressure to separate the membrane from bone and then uses mechanical pressure to pack the hard bone graft material thought the osteotomy in the lateral wall of the maxilla in order to raise the sinus membrane and fill the floor of the sinus with bone graft material. The current invention uses pneumatic pressure to separate the sinus membrane from bone and uses fluid bone graft under hydraulic pressure to lift the sinus membrane to the desired level in the desired direction.

### PRIOR ART

### Patents

U.S. Pat. No. 4,682,951
U.S. Pat. No. 4,787,848
U.S. Pat. No. 5,366,374
U.S. Pat. No. 5,456,601
U.S. Pat. No. 5,885,079
U.S. Pat. No. 5,989,025
U.S. Pat. No. 6,050,819
U.S. Pat. No. 6,537,070
2002/0177102

### OTHER REFERENCES

Artzi et al., “Nonceramic hydroxyapatite bone derivative in sinus augmentation procedures: clinical and histomorphometric observations in 10 consecutive cases,” Int. J. Periodontics Restorative Dent. 23(4):381-389, August 2003 (abstract only). cited by other.
Babbush, “Sinus lift revisited: an update on current implant-related procedures,” Dent. Implantol. Update 9(1):1-5, January 1998 (interview/abstract only). cited by other.
Baumann and Ewers, “Minimally invasive sinus lift. Limits and possibilities in the atrophic maxilla,” Mund Kiefer Gesichtschir 3 Suppl 1:S70-S73, May 1999 (abstract only). cited by other.
Boyne et al., “Grafting of the maxillary sinus floor with autogenous marrow and bone,” J. Oral Surg. 38:613-616, August 1980. cited by other.
Branemark et al., “An Experimental and Clinical Study of Osseointegrated Implants Penetrating the Nasal Cavity and Maxillary Sinus,” J. Oral Maxillofac Surg. 42(8):497-505, August 1984 (abstract only). cited by other.
Cehreli and Sahin, “Biological Reactions to a Poly(.sub.L-lactide)-Hydroxyapatite Composite: A Study in Canine Mandible,” J. Biomaterials Applications Applications 17:265-276, April 2003. cited by other.
Chen, “Hydraulic sinus lift with sinus condensers. Interview.” Dent. Implantol. Update 14(3):17-23, March 2003 (interview/abstract only). cited by other.
Coatoam, “Indirect sinus augmentation procedures using one-stage anatomically shaped root-form implants,” J. Oral Implantol. 23(1-2):25-42, 1997 (abstract only). cited by other.
Coatoam and Krieger, “A four-year study examining the results of indirect sinus augmentation procedures,” Oral Implantol. 23(3): 117-127, 1997 (abstract only). cited by other.
Cordaro, “Bilateral simultaneous augmentation of the maxillarysinus floor with particulated mandible. Report of a technique and preliminary results,” Clin. Oral Implants Res. 14(2):201-206, April 2003 (abstract only). cited by other.
Cosci and Luccioli, “A New Sinus Lift Technique in Conjunction With Placement of 265 Implants: A 6-Year Retrospective Study,” Implant Dent. 9(4):363-368, 2000 (abstract only). cited by other.
D'Amato et al., “Maxillary sinus surgical lift. Summers' technique versus lateral surgical approach,” Minerva Stomatol. 49(7-8):369-381, July-August 2000 (abstract only). cited by other.
Defrancq and Vanassche, “Less invasive sinus lift using the technique of Summers modified by Lazzara,” Rev. Belge Med. Dent. 56(2):107-124, 2001 (abstract only). cited by other.
Ellegaard et al., “Implant therapy involving maxillary sinus lift in periodontally compromised patients,” Clin. Oral Implants Res. 8(4):305-315, August 1997 (abstract only). cited by other.
Engelke and Deckwer, “Endoscopically controlled sinus floor augmentation. A preliminary report,” Clin. Oral Implants Res. 8(6):527-531, December 1997 (abstract only). cited by other.
Fugazzotto, “Sinus floor augmentation at the time of maxillary molar extraction technique and report of preliminary results,” Int. J. Oral Maxillofac Implants 14(4):536-542, July-August 1999 (abstract only). cited by other.
Gaggl et al., “Treatment planning for sinus lift augmentations through use of 3-dimensional milled models derived from computed tomography scans: a report of 3 cases,” Oral Surg. Oral Med. Oral Pathol. Oral Radiol. Endodi 86(4):388-392, October 1998 (abstract only). cited by other. 15(3):181-186, May-June 2001. cited by other.
Garg, “Augmentation grafting of the maxillary sinus for placement of dental implants: anatomy, physiology, and procedures,” Implant Dent. 8(1):36-46, 1999 (abstract only). cited by other.
Garg, “Nasal sinus lift: an innovative technique for implant insertions,” Dent. Implantol. Update 8(7):49-53, July 1997 (interview/abstract only). cited by other.
Gray et al., “Assessment of the sinus lift operation by magnetic resonance imaging,” Br. J. Oral Maxillofac Surg. 37(4):285-289, August 1999 (abstract only). cited by other.
Gray et al., “Magnetic resonance imaging assessment of a sinus lift operation using reoxidised cellulose (Surgicel) as graft material,” Clin. Oral Implants Res. 12(5)526-530, October 2001 (abstract only). cited by other.
Haas et al., “A preliminary study of monocortical bone grafts for oroantral fistula closure,” Oral Surg. Oral Med. Oral Pathol. Oral Radiol. Endod. 97(1):4, January 2004 (abstract only). cited by other.
Haas et al., “Bovine hydroxyapatite for maxillary sinus grafting: comparative histomorphometric findings in sheep,” Clin. Oral Implants Res. 9(2):107-116, April 1998 (abstract only). cited by other.
Haas et al., “Five-year results of maxillary intramobile Zylinder implants,” Br. J. Oral Maxillofac Surg. 36(2):123-128, April 1998 (abstract only). cited by other.
Haas et al., “Porous hydroxyapatite for grafting the maxillary sinus: a comparative histomorphometric study in sheep,” Int. J. Oral Maxillofac Implants 17(3):337-346, May-June 2002 (abstract only). cited by other.
Jian et al., “Maxillary pre-implant rehabilitation: a study of 55 cases using autologous bone graft augmentation,” Rev. Stomatol. Chir. Maxillofac. 100(5):214-220, October 1999 (abstract only). cited by other.
Johren et al., “Fear reduction in patients with dental treatment phobia,” Br. J. of Oral and Maxillofacial Surg. 38:612-616, 2000. cited by other.
Karabuda et al., “Histological and clinical evaluation of 3 different grafting materials for sinus lifting procedure based on 8 cases,” J. Periodontol. 72(10):1436-1442, October 2001 (abstract only). cited by other.
Kasabah et al., “Can we predict maxillary sinus mucosa perforation?” Acta Medica (Hradec Kralove) 46(1):19-23, 2003 (abstract only). cited by other.
Khoury, “Augmentation of the sinus floor with mandibular bone block and simultaneous implantation: a 6-year clinical investigation,” Int. J. Oral Maxillofac Implants 14(4):557-564, July-August 1999 (abstract only). cited by other.
Knabe and Hoffmeister, “The use of implant-supported ceramometal titanium prostheses following sinus lift and augmentation procedures: a clinical report,” Int. J. Oral Maxillofac Implants 13(1):102-108, January-February 1998 (abstract only). cited by other.
Krekmanov, “A modified method of simultaneous bone grafting and placement endosseous implants in the severely atrophic maxilla,” Int. J. Oral Maxillofac Implants 10(6):682-688, November-December 1995 (abstract only). cited by other.
Krekmanov and Heimdahl, “Bone Grafting to the maxillary sinus from the lateral side of the mandible,” British J. of Oral and Maxillofacial Surg. 38:617-619, 2000. cited by other
Lambrecht, “Intraoral piezo-surgery,” Schweiz Monatsschr Zahnmed. 114(1):28-36, 2004 (abstract only). cited by other.
Landi et al., “Maxillary sinus floor elevation using a combination of DFDBA and bovine-derived porous hydroxyapatite: a preliminary histologic and histomorphometric report,” Int. J. Periodontics Restorative Dent. 20(6):574-583, December 2000 (abstract only). cited by other.
Laster et al., “A new tricortical implant,” Refuat Hapeh Vehashinayim. 20(3):89-95, 105, July 2003 (abstract only). cited by other.
Leder et al., “Reconstruction of the severely atrophic maxilla with autogenous bone graft and hydroxylapatite/decalcified freeze-dried bone allograft in the same patient: a preliminary report,” Periodontal Clin. Investig. 15(1):5-9, Fall 1993 (abstract only). cited by other.
Lekholm, “New surgical procedures of the osseointegration technique A. M. Branemark,”Aust. Prosthodont J. 7 Suppl:25-32, 1993 (abstract only). cited by other.
Levin et al., “Smoking and complications of onlay bone grafts and sinus lift operations,” Int. J. Oral Maxillofac Implants 19(3):369-373, May-June 2004 (abstract only). cited by other.
Lim et al., “Intentional Angulation of an Implant to Avoid a Pneumatized Maxillary Sinus: A Case Report,” J. Canadian Dental Assoc. 70(3):164-168, March 2004. cited by other.
Marx and Garg, “A novel aid to elevation of the sinus membrane for the sinus lift procedure,” Implant Dent. 11(3):268-271, 2002 (abstract only). cited by other.
Mazor et al., “The use of hydroxyapatite bone cement for sinus floor augmentation with simultaneous implant placement in the atrophic maxilla. A report of 10 cases.” J. Periodontol. 71(7):1187-94, July 2000 (abstract only). cited by other.
Murakami et al., “Periodontal and computer tomography scanning evaluation of endosseous implants in conjunction with sinus lift procedure. A case series,” J. Periodontol. 70(10):1254-1259, October 1999 (abstract only). cited by other.
Muronoi et al, “Simplified procedure for augmentation of the sinus floor using a haemostatic nasal balloon,” Br. J. Oral Maxillofac Surg.41(2):120-121, April 2003 (abstract only). cited by other.
Nedir et al., “A 7-year life table analysis from a prospective study on ITI implants with special emphasis on the use of short implants. Results from a private practice.” Clin. Oral Implants Res. 15(2):150-157, April 2004 (abstract only). cited by other.
Nocini et al., “Implant placement in the maxillary tuberosity: the Summers' technique performed with modified osteotomes” Clin. Oral Impl. Res. 11:273-278, 2000. cited by other.
Olson et al., “Long-term assessment (5 to 71 months) of endosseous dental implants placed in the augmented maxillary sinus,” Ann Periodontol. 5(1):152-156, December 2000 (abstract only). cited by other.
Pacifici et al., “Lifting of the maxillary sinus: complementary use of platelet rich plasma, autologous bone deproteinised bovine bone. Case report,” Minerva Stomatol 52(9):471-478, September 2003 (abstract only). cited by other.
Partridge and Hostalet, “New harvesting technique for bone grafting. Case reports.” J. Indiana Dent. Assoc. 82(2):19-22, Summer 2003 (abstract only). cited by other.
Peleg et al., “Radiological findings of the post-sinus lift maxillary sinus: a computerized tomography follow-up,” J. Periodontol. 70(12):1564-1573, December 1999 (abstract only). cited by other.
Rosen et al., “The bone-added osteotome sinus floor elevation technique: multicenter retrospective report of consecutively treated patients,” Int. J. Oral Maxillofac Implants 14(6):853-858, November-December 1999 (abstract only). cited by other.
Rosenlicht and Tarnow, “Human histologic evidence of integration of functionally loaded hydroxyapatite-coated implants placed simultaneously with sinus augmentation: a case report 2½ years postplacement,” J. Oral Implantol. 25(1):7-10, 1999 (abstract only). cited by other.
Scher, “Use of the incisive canal as a recipient site for root form implant: preliminary clinical reports,” Implant Dent. 3(1):38-41, Spring 1994 (abstract only). cited by other.
Shiota et al., “Clinical retrospective study on outpatients at clinic for oral implants,” Kokubyo Gakkai Zasshi 66(1):15-19, March 1999 (abstract only). cited by other.
Smiler et al., “Sinus lift grafts and endosseous implants. Treatment of the atrophic posterior maxilla,” Dent. Clin. North Am. 36(1):151-186, January 1992. cited by other.
Soltan and Smiler, “Trephine bone core sinus elevation graft,” Implant Dent. 13(2):148-152, June 2004 (abstract only). cited by other.
Sulzer et al., “Indications for oral implantology in a referral clinic. A three-year retrospective analysis of 737 patients with 1176 implants,” Schweiz Monatsschr
Zahnmed. 114(5):444-450, 2004 (abstract only). cited by other.
Summers, “Sinus Floor Elevation with Osteotomes,” J. Esthet. Dent. 10(3):164-171, 1998 (abstract only). cited by other.
Szabo, “Use of biomaterials for preventive and restorative purposes,” Fogorv Sz. 85(3):65-69, March 1992 (abstract only). cited by other.
Tepper et al., “Effects of sinus lifting on voice quality,” Clin. Oral Impl. Res. 14:767-774, 2003. cited by other.
Thunthy et al., “An illustrative study of the role of tomograms for the placement of dental implants,” J. Oral Implantol. 29(2):91-95, 2003 (abstract only). cited by other.
Tong et al., “A review of survival rates for implants placed in grafted maxilla sinuses using meta-analysis,” Int. J. Oral Maxillofac Implants 13(2):175-182, March-April 1998 (abstract only). cited by other.
Vachiramon et al., “Delayed immediate single-step maxillary sinus lift using autologous fibrin adhesive in less than 4-millimeter residual alveolar bone: a case report,” J. Oral Implantol. 28(4):189-193, 2002 (abstract only). cited by other.
van den Bergh et al., “Anatomical aspects of sinus floor elevations,” Clin. Oral Implants Res. 11(3):256-265, June 2000 (abstract only). cited by other.
Vassos and Petrik, “The sinus lift procedure: an alternative to the maxillary subperiosteal implant,” Pract. Periodontics Aesthet. Dent. 4(9):14-19, November-December 1992 (abstract only). cited by other.
Weingart and Ten Bruggenkate, “Treatment of fully edentulous patients with ITI implants,” Clin. Oral Implants Res. 11 Suppl 1:69-82, 2000 (abstract only). cited by other.
Wheeler et al., “Six-year clinical and histologic study of sinus-lift grafts,” Int. J. Oral Maxillofac Implants 11(1):26-34, January-February 1996 (abstract only). cited by other.
Woo and Le, “Maxillary Sinus Floor Elevation: Review of Anatomy and Two Techniques,” Implant Dentistry 13(1):28-32, 2004. cited by other.
Xu et al., “Experimental sinus grafting with the use of deproteinized bone particles of different sizes,” Clin. Oral Implants Res. 14(5):548-555, October 2003 (abstract only). cited by other.
Zhao et al., “Maxillary sinus lifting and simultaneously placement of implants from the top of alveoli,” Zhonghua Kou Qiang Yi Xue Za Zhi 38(4):251-253, July 2003 (abstract only). cited by other.
Zide, “Autogenous bone harvest and bone compacting for dental implants,” Compend. Contin. Educ. Dent. 21(7):585-590; quiz 592, July 2000 (abstract only). cited by other.
Zinner et al., “Provisional and definitive prostheses following sinus lift and augmentation procedures,” Implant Dent. 3(1):24-28, Spring 1994 (abstract only). cited by other.
Zinner and Small, “Sinus-lift graft: using the maxillary sinuses to support implants,” J. Am. Dent. Assoc. 127(1):51-57, January 1996 (abstract only). cited by other.
Woo and Le, “Maxillary Sinus Floor Elevation: Review of Anatomy and Two Techniques,” Implant Dentistry 13(1):28-32, 2004. cited by other.
Xu et al., “Experimental sinus grafting with the use of deproteinized bone particles of different sizes,” Clin. Oral Implants Res. 14(5):548-555, October 2003 (abstract only). cited by other.
Zhao et al., “Maxillary sinus lifting and simultaneously placement of implants from the top of alveoli,” Zhonghua Kou Qiang Yi Xue Za Zhi 38(4):251-253, July 2003 (abstract only). cited by other.
Zide, “Autogenous bone harvest and bone compacting for dental implants,” Compend. Contin. Educ. Dent. 21(7):585-590; quiz 592, July 2000 (abstract only). cited by other.
Zinner et al., “Provisional and definitive prostheses following sinus lift and augmentation procedures,” Implant Dent. 3(1):24-28, Spring 1994 (abstract only). cited by other.
Zinner and Small, “Sinus-lift graft: using the maxillary sinuses to support implants,” J. Am. Dent. Assoc. 127(1):51-57, January 1996 (abstract only). cited by other.

### SUMMARY OF THE INVENTION

This invention is a method of growing bone in the maxillary sinus. A small incision is made to gain access to the lateral wall of the maxilla in the area of the posterior maxillary alveolar bone. A small hole is drilled to access the sinus. Pneumatic pressure is used to safely and easily detach the sinus membrane from the lateral wall of the maxillary sinus. After the membrane is detached a fluid bone graft material is injected lifting the membrane lining of the maxillary sinus by hydraulic pressure.

### DETAILED DESCRIPTION OF THE INVENTION

This invention is a method of regenerating bone in the maxillary sinus. Due to the pneumatization of the sinus the lower portion of the maxillary sinus cavity is in close proximation to the crest of the posterior maxillary alveolar ridge. A minor incision is made in the buccal mucosa or buccal attached gingiva of the maxillary posterior alveolar ridge near the lowest point of the sinus as read by radiographic evaluation. After exposing the lateral wall of the maxilla in the area of the low point of the sinus a small hole is drilled to gain access to the sinus membrane. The hole need only be large enough to access the sinus membrane. The thickness of bone is usually 1-3 mm in this area of the maxilla. As the sinus is dark and bone is white and the osteotomy is only 1-3 mm deep the sinus membrane can be seen before the membrane is exposed. Knowing that the membrane is near the surface and being able to see the location of the membrane prevents damage to the membrane as it is exposed. Because the graft material is fluid the diameter of the osteotomy need only be between 2-3 millimeteres in diameter and can be injected with a syringe. After exposure of the sinus membrane air pressure is applied to the membrane to gently detach it from the internal aspect of the lateral wall of the maxillary sinus. After pneumatically detaching the sinus membrane it is checked to ensue the membrane is free from the floor of the sinus and intact. Fluid bone graft material is injected through the small osteotomy in the lateral wall of the maxillary sinus. The hydraulic pressure created between the bone of the maxillary sinus and the sinus membrane by injecting a fluid bone graft material lifts the sinus membrane. Bone graft material is injected until the sinus membrane is lifted to the desired level. In time the bone graft material is converted into the patients own bone thereby reducing the size of the sinus and increasing the amount of alveolar bone available for implant placement in the posterior maxilla. Pneumatic pressure applied to the membrane safely detaches the membrane without damage. Introducing the graft material under hydraulic pressure permits sterile technique and lifts the membrane to any level as determined by the amount of graft material injected through a minimally invasive osteotomy.

The advantages of this invention include:
Minimally invasive surgical procedure.
The osteotomy is minimal being 1-3 mm deep and wide.
The dark sinus can be seen prior to reaching the membrane.
The membrane is easily visualized assuring detachment without damage.
Pneumatic pressure detaches the membrane from bone safely and without filling the sinus with water.
Directed hydraulic pressure lifts the membrane in the desired direction.
The amount of lift is determined by the volume of graft injected.
Lifting the membrane with hydraulic pressure prevents membrane damage.
The graft material sets hard and supports implants placed in minimal bone.
No minimal amount of bone needed for immediate implant placement.
The graft material is osteogenic and quickly resorbed.
Implant loading advised in 3 months.
Minimal instrumentation with closed graft deliver permits a sterile technique.
Simplicity of the procedure requires less time and expertise.

This invention significantly reduces trauma to the patient thereby reduces post operative morbidity. This invention reduces the amount of surgical expertise required to perform a sinus lift thereby making the procedure available to the public by more practitioners. This invention reduces the amount of time to perform a sinus lift thereby reducing the cost and making the procedure more available to the public. This invention prevents damage to the sinus membrane thereby making sinus lift surgery more predictable with fewer complications.

1. A method of detaching a sinus membrane from it's supporting bone by preparing an osteotomy adjacent to the sinus membrane and obturating the osteotomy with a device that delivers pneumatic pressure in a mammal in need thereof.

2. The method of claim 1 where space is created between a sinus membrane and it's supporting bone in a mammal in need thereof.

3. A method of regenerating bone in a sinus by preparing an osteotomy adjacent to the sinus membrane and obturating the osteotomy with a device that deposits a fluid bone graft by way of hydraulic pressure in a mammal in need thereof.

4. The method of claim 3 where the hydraulic pressure created by injecting a fluid bone graft between a sinus membrane and the bone that supports the sinus membrane lifts the sinus membrane.
